(19) United States
(12) Patent Application Publication
(10) Pub. No.: US 2016/0051586 A1
(43) Pub. Date: Feb. 25, 2016

(54) METHODS OF GROWING AND PREPARING STEM CELLS AND METHODS OF USING THE SAME

(71) Applicant: JEREMY DELK, Nicholasville, KY (US)

(72) Inventor: JEREMY DELK, Nicholasville, KY (US)

(21) Appl. No.: 14/832,625

(22) Filed: Aug. 21, 2015

Related U.S. Application Data
(60) Provisional application No. 62/040,149, filed on Aug. 21, 2014.
(60) Provisional application No. 62/040,153, filed on Aug. 21, 2014.
(60) Provisional application No. 62/040,170, filed on Aug. 21, 2014.

(51) Int. Cl. A61K 35/28; A61K 35/19; C12N 5/0775; A61K 35/16

(52) U.S. Cl. CPC A61K 35/28; A61K 35/16; A61K 35/19; C12N 5/0662; C12N 2531/00

(57) ABSTRACT

The present invention relates to methods of improving stem cell delivery to a subject in need thereof and kits designed to assist in such. The methods comprise interchangeably allowing or promoting cell growth in conditions that permit three-dimensional growth, such as with a bioreactor, utilizing allogeneic, autologous or xenogeneic cells, mixing the cells with platelet rich plasma that is autologous, allogeneic or xenogeneic to the subject, and site specific delivery of between about three and ten million activated stem cells per kilogram of the subject receiving the treatment.

### RELATED APPLICATIONS

This patent application claims priority to U.S. Provisional Patent Application Nos. 62/040,149, filed on Aug. 21, 2014, 62/040,153, filed on Aug. 21, 2014, and 62/040,170, filed on Aug. 21, 2014, the entireties of which are incorporated herein by reference.

### FIELD OF THE INVENTION

The present invention relates generally to methods of growing and using stem cells for medical applications.

### BACKGROUND

Recently, it has been proposed to use stem cells to treat bone, ligament, tendon or cartilage injury (see U.S. patent application Ser. No. 13/773,869, incorporated herein by reference in its entirety). Stem cells may be derived from a variety of sources including adipose tissues from the Stromal Vascular Fraction (SVF), bone marrow, the umbilical cord, and blood. Additionally, adipose-derived stem cells (ADSCs) or adipose-derived mesenchymal stem cells (ADMSCs) have been shown to possess the ability to generate multiple tissues, including bone, fat, cartilage, and muscle despite being in an “inactive” state when extracted. As set forth in U.S. patent application Ser. No. 13/77,869, these stem cells can be activated by photo-activation and/or contact with platelet rich plasma (PRP). Thus, adipose tissue has been proposed as an optimal source for adult stem cells (ASCs) for use in regenerative medicine. But, what are needed in the art are improved methods for growing and administering the stem cells.

### SUMMARY OF THE INVENTION

The present invention provides in part methods of growing autologous, allogeneic or xenogeneic stem cells, comprising providing a biologically active, three-dimensional environment for said cells; and culturing said stem cells in said environment. The cells can be delivered to the patient suspended in platelet rich plasma autologous to the subject to which the cells are to be administered. The environment for incubating the cells may comprise a bioreactor vessel and an enriched medium. The present invention provides a method of treating a patient, comprising administering cells grown in a 3D environment in conjunction with delivery to the patient suspended in autologous platelet rich plasma. 3D cultured cells may be combined with autologous stem cells from the patient to receive the cells.

The present invention also provides a method of treating a patient, comprising administering autologous, allogeneic or xenogeneic stem cells grown in a 3D environment in conjunction with delivery of the cells suspended in photo bio-stimulated platelet rich plasma. For site specific delivery, the total number of cells administered is between about 5 and 15 million cells. System administration requires administration of between 3 and 10 million cells per kilogram of the subject. The platelet rich plasma can be allogeneic, autologous or xenogeneic. As described herein, expanding or growing in a 3D environment can be achieved by incubating cells in a bioreactor.

In accordance with the purposes described herein, a method is provided for growing autologous, allogeneic or xenogeneic stem cells. The method may be broadly described as growing/culturing stem cells in a biologically active, three-dimensional environment, such as a bioreactor.

In accordance with an additional aspect, a method is provided for administering the cells in conjunction with autologous platelet rich plasma (PRP) to a patient.

In accordance with yet another aspect, a method is provided for administering the cells in conjunction with autologous, photobiostimulated PRP to a patient.

### DESCRIPTION

### Stem Cell Administration

The present invention provides for administering stem cells to a patient or a subject. A subject may be a human, canine, feline, bovine, ovine, equine or porcine or a zoo animal. In certain situations, the stem cells are autologous to the patient receiving them. However, certain aspects of the present invention provide methods for preparing allogeneic or xenogeneic stem cells that are administered to the patient or subject. The methods of the present invention have identified particular growth/culturing conditions that allow for beneficial administration of stem cells. The method may be broadly described as growing/culturing stem cells in a biologically active, three-dimensional environment, such as a bioreactor.

Autologous and allogeneic cells (or donor cells or non-autologous cells) refer to cells that are genetically different but belong to or are obtained from the same species; autologous cells (or patient cells) are cells that are genetically the same or derived from the same subject or the same subject's same tissue. Xenogeneic cells refer to cells from a different species. Stem cells can be deemed allogeneic when administered to a genetically different environment from the source of the cells, such as that of a different patient or subject. Stem cells can be collected and concentrated as described in U.S. patent application Ser. No. 13/773,869 and thereafter, concentrated stem cells may be further activated with isolated platelet rich plasma (PRP) to the patient or subject and may be photo-biostimulated. For the purposes described herein, stem cells may refer to stem cells that are pre-treated with PRP that is optionally photo-activated, as well as naïve concentrated stem cells. As described in U.S. patent application Ser. No. 13/773,869, PRP can optionally be prepared from the same sample from which the stem cells are concentrated. As described herein, with the use of autologous or allogeneic bioreactor-expanded stem cells, the PRP can be autologous to the patient receiving the stem cells. 3D cultured cells can be cultured in allogeneic or autologous PRP. 3D cultured cells can then be combined with further autologous stem cells as described in U.S. patent application Ser. No. 13/773,869 that further comprise autologous PRP with further optional photo-biostimulation.

In order to prepare stem cells for administration, a collected fat or other tissue sample is treated to isolate the stem cells and then they are grown or cultured so that the stem cells interact/grow in three dimensions. Three-dimensional (3D) cell culturing methods are understood in the art and can include methods associated with apparatuses such as extracellular scaffolds, bioreactors, micro-carriers, magnetic levitation, hanging drop plates, magnetic bio-printing and modified surfaces. For example 3D culturing of stem cells can occur when the cells are conditioned in a bioreactor. A bioreactor is any manufactured or engineered device or system that supports a biologically active environment. In some instances, the bioreactor is a vessel in which a chemical/biochemical process is carried out which involves contact with organisms or biochemically active substances derived from such organisms. Such processes can either be aerobic or made anaerobic. Such bioreactors are commonly cylindrical, ranging in size from 2 liters to 10000 liters, and often made of stainless steel. A bioreactor may also refer to a device or system meant to grow cells or tissues in the context of cell culture. Such devices are seen in tissue engineering or biochemical engineering (see, e.g., John W. Haycock (ed.), 3D Cell Culture: Methods and Protocols, Methods in Molecular Biology, vol. 695, DOI 10.1007/978-1-60761-984-0_{—}1, Springer Science+Business Media, LLC 2011).

Compared to cells grown in two-dimensional (2D) cultures, the use of three-dimensional (3D) cell cultures provides a closer mimicking of natural tissues and organs. In 2D cultures, cells are grown on flat dishes made of polystyrene plastic that is very stiff and unnatural. The cells adhere and spread on the plastic surface and form unnatural cell attachments to proteins that are deposited and denatured on the surface. In 3D cultures, cells attach to a microcarrier and form natural, cell-to-cell attachments. The cells and the extracellular matrix that they synthesize and secrete in three dimensions is the natural material to which cells are attached. It is made of complex proteins in their native configuration and so provides important biological instructions to the cells. In this 3D cell culture environment, cells can exert forces on one another and can move and migrate as they do in vivo. These cell-to cell interactions in three dimensional cell culture also include gap junctions which directly couple one cell to another.

The gap junctions are much more prevalent in 3D cell culture than 2D and these junctions enable cells to communicate with each other via exchange of ions, small molecules, and even electrical currents. The close proximity of cells in 3D also enable surface adhesion molecules and surface receptors on one cell to bind to surface adhesion molecules and surface receptors on an adjacent cell. This coupling in 3D also maximizes cell-to-cell communication and signaling that is critical for cell function. Not surprisingly, the phenotype or function of cells grown in 3D is more complex and closer to the functions of native tissues than cells grown in 2D. Liver cells will perform more liver cell functions in 3D versus 2D. Muscle cells will perform more muscle cell functions in 3D cell culture versus 2D cell culture. Cartilage cells will form more differentiated cartilage tissue in 3D versus 2D. And the list continues with nearly all the cells of solid organs and tissues.

Cells can be optionally cultured in PRP and/or photo-biostimulated in order to assist in activation. Following 3D growth of the stem cells, cells can be isolated and optionally mixed with derived autologous stem cells from the patient as previously described. 3D cultured stem cells, with or without autologous stem cells (such ADSC), can be administered to the patient. The autologous stem cells that are mixed in may be photo-activated and or mixed with autologous PRP to assist in activating these cells. PRP isolated from the subject to receive the stem cells (optionally photo-activated) can be mixed with the mixed 3D and/or autologous stem cells and then administered to the subject. The stem cells may be administered systemically, such as by i.v., or site-specifically, such as by i.a. For site specific delivery, the total number of cells administered is between about 5 and 15 million cells. System administration requires administration of between about 3 and 10 million cells per kilogram of the subject.

The administered cells may comprise a specific dose or number of stem cells. The dosage of cells may be from about 3 to about 10 million cells per kilogram when administered systemically and between 5 and 15 million cells when applied to a particular site, such as a joint, tendon, bone, or organ. The cells may be administered intra-articularly, intravenously, intramuscularly, intraperitoneally, topically or by other routes known in the art.

### KITS

The present invention also provides for kits for executing the methods described herein. The kits may include: devices for collecting a sample of tissue/blood from which stem cells may be obtained and concentrated, a device for promoting 3D growth of a stem cell, such as a bioreactor, antibiotics, antifungals, a device for administering concentrated stem cells, a device for isolating PRP, a photo bio-stimulator and a device for concentrating stem cells.

### EXAMPLES

### Bioreactor

### Methods

Twenty 250 ml scale pilot runs using six MSC isolates were performed to optimize conditions for large scale (2.4 L) bioreactor trial. For the large scale bioreactor trial, human MSCs were plated at 8000 cells per cm² on plastic microcarriers (Plastic Plus supplied by SoloHill) in DMEM supplemented with pooled human platelet lysate. The microcarrier suspension was transferred to a Millipore Mobius 3 L single use bioreactor which was controlled by an Applikon EZ controller. The temperature was set to 37° C. and maintained by a heating jacket, the pH was regulated at pH 7.4 by sparging with CO₂ to lower pH or by addition of 1N NaOH to raise pH. The dissolved oxygen was set to 40% and maintained by sparging with air. The stirring speed was initially set to 70 rpm and this speed was increased to 120 rpm on day 4. To perform medium replacements, the stirred was turned off and the carriers allowed to settle down as much as possible. Then most of the medium solution was decanted from the bioreactor. To assess growth rate and metabolites, twice a day 3 ml samples were collected from the fluid column. The unattached cells and microcarriers were stained with Sybr-green to evaluate attachment and confluence of carriers. The cells were removed from the microcarriers by exposure to trypsin for 15 min and then the cells were separated from the microcarriers by filtration. The cells were pelleted by centrifugation, stained to determine live/dead ratio and number of cells was determined using the Nexcelom Auto 2000 cell viability counter.

To confirm MSC status, the surface expression of cells from the bioreactor was evaluated for presence of mesenchymal markers (positive markers) and absence of hematopoietic cells (negative markers). Additionally, the MSCs were shown to be multipotent by differentiation to osteogenic, chondrogenic or adipogenic lineages by exposure to the StemPro differentiation kit (from Life Technologies).

### RESULTS

After testing five microcarrier types, Plastic Plus microcarriers from SoloHill were found to have the best attachment (75.3% occupancy) and the highest cell number at confluence (1.1E4 cells/cm²). Growth kinetics were determined to be 18.9 hr doubling time and 14.8 fold expansion on average (n=5). Glucose consumption was found to be an average of 0.012 gram/L*h, glutamine consumption was found to be 0.032 mM/hr, lactate accumulation was found to be 0.020 g/L*h, and ammonium accumulation was found to be 0.016 mM/h.

In summary, 3D culture was found to have a longer lag phase and a greater overall yield of cells at harvest compared to 2D culture.

All publications, patents and patent applications references herein are to be each individually considered to be incorporated by reference in their entirety.

What is claimed:

1. A method of treating a site specific injury in a subject comprising administration of a suspension of stem cells to a site in need thereof, wherein the suspension comprises about 3-ten million activated stem cells per kilogram of the subject intravenously or about 5 to 10 million cells intra-articularly and further wherein the stem cells are derived from a 3D cell culture.

2. The method of claim 1, wherein the stem cells comprise autologous, allogeneic or xenogeneic cells incubated in a bioreactor prior to administration to the subject.

3. The method of claim 1, wherein the suspension further comprises a bio-compatible matrix.

4. The method of claim 1, further comprising mixing the stem cells with autologous platelet rich plasma prior to administration to the subject.

5. The method of claim 4, wherein the autologous PRP is photo bio-stimulated.

6. The method of claim 1, further comprising mixing the suspension with autologous adipose-derived stem cells (ADSCs) isolated from the subject prior to administration.

7. The method of claim 6, wherein the autologous ADSCs are previously activated by PRP and/or photo-biostimulation.

8. A method of treating a patient, comprising administering a suspension of stem cells grown to the patient, wherein the suspension of stem cells are derived from a 3D cell culture and contacted with platelet rich plasma and/or photo bio-stimulated to the patient.

9. The method of claim 8, further comprising adding autologous ADSC to the suspension.

10. The method of claim 9, wherein the autologous ADSC are photo-biostimulated.

11. The method of claim 9, wherein the autologous ADSC are contacted with PRP.

12. The method of claim 6, wherein the stem cells are administered at a count of between about 5 to 15 million cells intra-articularly at a joint, tendon or bone in need thereof.

13. The method of claim 6, wherein the stem cells are administered at a count of between about 3 to 10 million cells per kilogram of the patient intravenously.
